(19) United States
(12) Patent Application Publication
(10) Pub. No.: US 2024/0103285 A1
(43) Pub. Date: Mar. 28, 2024

(54) INTEGRATED HEALTH SENSORS

(71) Applicant: Apple Inc., Cupertino, CA (US)

(72) Inventors: Javier Mendez, San Jose, CA (US); Grant H. Mulliken, Los Gatos, CA (US); Trevor J. Ness, Santa Cruz, CA (US); Samuel G. Smith, San Francisco, CA (US); Xinsheng Chu, Saratoga, CA (US)

(21) Appl. No.: 18/460,224

(22) Filed: Sep. 1, 2023

Related U.S. Application Data
(60) Provisional application No. 63/376,280, filed on Sep. 19, 2022.

(51) Int. Cl. G02B 27/01; A61B 5/00; A61B 5/0205; G06V 40/16

(52) U.S. Cl. CPC G02B 27/0176; A61B 5/02055; A61B 5/6803; A61B 5/74; G06V 40/161; G06V 40/174

(57) ABSTRACT

A head-mountable device including a frame, a display positioned in the frame, a processor, and a facial interface connected to the frame. The facial interface can include a sensor electrically coupled to the processor. The sensor can collect biometric information from a nasal region of a user and generate a signal based on the biometric information.

### CROSS-REFERENCE TO RELATED APPLICATION(S)

This claims priority to U.S. Provisional Patent Application No. 63/376,280, filed 19 Sep. 2022, and entitled “Integrated Health Sensors,” the entire disclosure of which is hereby incorporated by reference.

### FIELD

The described embodiments relate generally to facial interfaces for a head-mountable devices. More particularly, the present embodiments relate to facial interfaces for head-mountable device including health sensors.

### BACKGROUND

Recent advances in portable computing have enabled head-mountable devices (HMD(s)) that provide augmented and virtual reality (AR/VR) experiences to users. The ever-increasing complexity and functionality of these head-mountable devices encourages constant improvements to the electrical components and sensors of the HMD.

Sensors can be utilized on head-mountable devices for various purposes, such as detecting user information, user identity, biometric information, environmental detection, movement, location, etc. An arrangement of sensors that comports with the structure, materials, and purpose of the sensor and head-mountable device is needed.

Unfortunately, sensors in conventional head-mountable devices are employed in rudimentary ways, if at all, leading to a limited user experience, and creating user discomfort and dissatisfaction. Indeed, sensors in conventional head-mountable devices can lend to bulky, heavy, and/or cumbersome devices, whose sensors are ineffective at realizing their full potential.

### SUMMARY

According to some aspects of the present disclosure, a head-mountable device can include a frame, a display positioned in the frame, a processor, and a facial interface connected to the frame. The facial interface can include a sensor electrically coupled to the processor. The sensor can collect biometric information from a nasal region of a user and can generate a signal based on the biometric information.

In some examples, the sensor can transmit the signal to the processor. The processor can analyze the signal and can cause the head-mountable device to perform an action in response to the signal. The action can include providing at least one of a visual feedback, an audio feedback, or a haptic feedback. The sensor can include at least one of a temperature sensor, a respiration sensor, a stress response sensor, or a heart activity sensor.

In some examples, the processor can determine a facial expression of the user based on the signal. The facial interface can be removably attached to the frame. The sensor can be removably attached to the facial interface. The facial interface can be in electrical communication with the display. In some examples, the head-mountable device can include a retention band, wherein the facial interface includes a connector to attach to the retention band.

According to some aspects, a facial interface for a head-mountable device can include a first side having a connector to attach the facial interface to a display, and a second side to contact a face of a user. A sensor of the facial interface can detect a biometric feature of the user, wherein the sensor can be positioned proximate a nose of the user, and wherein the biometric feature comprises a facial expression of the user.

In some examples, the sensor can be embedded in the facial interface. The second side of the facial interface can include an area transparent to a signal emitted by the sensor. The facial interface can include a controller. The controller can include a processor to receive biometric data from the sensor and analyze the biometric data, and a memory device storing computer-executable instructions that, when executed by the processor, can cause a component of the head-mountable device to perform an action in response to the signal. In some examples, the display can perform a function based on the detected biometric feature.

According to some examples, a wearable electronic device can include a display, an engagement interface, and a sensor coupled to the engagement interface. The sensor can detect a biometric feature and produce a signal based on the biometric feature. A processor can analyze the signal. In response to the signal, the wearable electronic device can perform an action.

In some examples, the action can include providing a notification to a user. The wearable electronic device can include a head-mountable device. The engagement interface can be adjustable. The biometric feature can include a feature of the autonomic nervous system.

The sensor can include a first sensor oriented towards a first facial region. A second sensor can be coupled to the engagement interface, the second sensor oriented towards a second facial region different from the first facial region when the wearable electronic device is worn. The engagement interface can change shape in response to the signal from the sensor. The engagement interface can be a first engagement interface. The sensor can be a first sensor. The display can be removably attachable to the first engagement interface and a second engagement interface having a second sensor.

### BRIEF DESCRIPTION OF THE DRAWINGS

The disclosure will be readily understood by the following detailed description in conjunction with the accompanying drawings, wherein like reference numerals designate like structural elements.

FIG. 1 shows a block diagram of a head-mountable device.

FIG. 2 shows a top view of an example head-mountable device.

FIG. 3 shows a rear perspective view of an example head-mountable device including a facial interface incorporated with sensors.

FIG. 4 shows a cross-sectional view of a facial interface with sensors disposed at various locations.

FIG. 5 shows a perspective view of a head-mountable device including sensors.

FIG. 6 shows a perspective view of a head-mountable device including a facial interface, a frame, and a plurality of electronic components.

FIG. 7 shows a lower perspective view of an example of a sensor system for a head-mountable device.

### DETAILED DESCRIPTION

Representative embodiments illustrated in the accompanying drawings are detailed below. The following descriptions are not intended to limit the embodiments to one preferred embodiment. Rather, they are intended to cover alternatives, modifications, and equivalents as can be included within the spirit and scope of the described embodiments as defined by the appended claims.

The following disclosure relates to a facial interface of a head-mountable device. More particularly, the present embodiments relate to a facial interface of a head-mountable device including health sensors used for acquiring biometric information of the user, and performing actions in response to the detected biometric information. These facial interfaces can enable health sensors to interact with a user to collect biometric information. In some examples, a processor causes a component of the HMD to perform an action in response to the biometric information collected by the sensor. As used herein “an action” or “perform an action” can refer to any electrical or mechanical act the causes a change in one or more components of the HMD. For example, the action can include displaying a notification for the user, or activating or deactivating one or more components. In some examples, a sensor can collect biometric information of a user who is exercising. The biometric information can be related to temperature, respiration, stress response, heart activity, or brain activity or any other pertinent data. Further, in response to the collected data, they system can perform an action (e.g., notify the user of the collected data.

Head-mountable devices equipped with sensors are utilized for various purposes that detect limited user feedback, such as movement or positioning feedback, providing limited information in response to the user. For example, a user may have an increase in heartrate or brain activity while performing an action or movement. Conventional head-mountable devices are not suitably equipped to capture all the necessary biometric information generated during use.

In contrast, the head-mountable devices of the present disclosure include a facial interface that can be integrated or incorporated with sensors configured to collect user data, such as biometric information, including heartrate, respiration, brain activity, etc. By capturing the user data, the facial interface sensors can provide improved and increased feedback to the user. A head-mountable device with sensors monitoring user biometric or health feedback can create highly customized user experiences, unlike the sensors of conventional head-mountable devices that are unable to consider, react to, or further enhance the user experience.

Sensors positioned on the facial interface are important to create a customized user experience. The head-mountable device of the present disclosure can contain sensors to measure a user's response or engagement via indicators, such as heart rate, electrical signals from the heart (e.g., ECG, EKG, EXG, etc.), brain activity (e.g., EEG signals, frontal lobe activity), core body temperature, etc. Additionally, the sensor data can be used as feedback data, for example, to monitor user fatigue, facial expressions, or obtain activity-specific metrics.

The head-mountable device can have different sensors implemented in different ways. For example, the head-mountable device of the present disclosure can implement sensors removably attached to the facial interface. In some examples, the removably attached sensors correspond to a different user activity, such as exercise, learning activities, health, clinical settings, etc. In certain implementations, the exercise sensors can include a different sensor, such as a heart rate monitor sensor, whereas the learning activity sensor may include a brain activity sensor. The sensors can be implemented in different applications and arrangement/combinations for obtaining different, activity biometric readings.

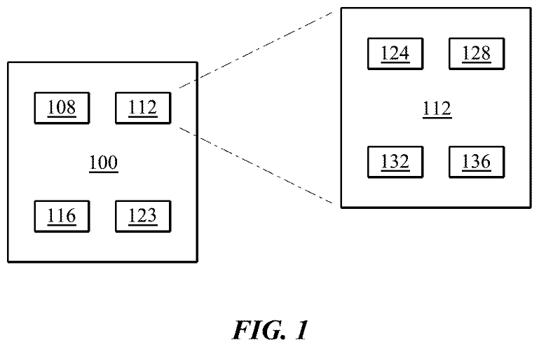

These and other embodiments are discussed below with reference to FIGS. 1-6. However, those skilled in the art will readily appreciate that the detailed description given herein with respect to these Figures is for explanatory purposes only and should not be construed as limiting. Furthermore, as used herein, a system, a method, an article, a component, a feature, or a sub-feature comprising at least one of a first option, a second option, or a third option should be understood as referring to a system, a method, an article, a component, a feature, or a sub-feature that can include one of each listed option (e.g., only one of the first option, only one of the second option, or only one of the third option), multiple of a single listed option (e.g., two or more of the first option), two options simultaneously (e.g., one of the first option and one of the second option), or combination thereof (e.g., two of the first option and one of the second option).

FIG. 1 illustrates a block diagram of a head-mountable device 100 including a frame 116, a display 108, a facial interface 112, and a support or retention band 123. The display 108 can include one or more optical lenses or display screens in front of the eyes of a user. The display 108 can include a display or display unit for presenting an augmented reality visualization, a virtual reality visualization, or other suitable visualization to a user. Additionally, the display 108 can be positioned in or on the frame 116. Similarly, a facial interface 112 can be connected to the frame 116. In some examples, the frame 116 can be a housing for the display 108. Alternatively, a separate housing can form an exterior portion of the head-mountable device 100.

The facial interface 112 can include one or more sensors 124, support attachments 132, display attachments 128, and feedback or output modules 136. The sensors 124 can be removably attached to the facial interface 112. As used herein, the terms “facial interface”, “engagement interface”, or “light seal” refer to a portion of the head mountable device 100 that engages (i.e., contacts or conforms to) a user's face. In particular, the facial interface 112 can include portions of a head-mountable device that conform or press against regions of a user's face. The facial interface 112 can be positioned between the display 108 and the user's face. In some examples, the facial interface 112 can include a pliant (or semi-pliant) facetrack or lumen that spans the forehead, wraps around the eyes, contacts other regions of the face (etc., zygoma and maxilla regions), and bridges the nose.

In addition, the facial interface 112 can include various components forming a frame, structure, or webbing of a head mountable device disposed between the display 108 and the user's skin. In particular implementations, the facial interface 112 can include a seal (e.g., a light seal, environment seal, dust seal, air seal, etc.). It will be appreciated that the term “seal” can include partial seals or inhibitors, in addition to complete seals (e.g., a partial light seal where come ambient light is blocked and a complete light seal where all ambient light is blocked when the head-mountable device 100 is donned). The facial interface 112 can be removably attached to the frame 116 and in electrical communication with the display 108.

The sensor 124 of the facial interface 112 can collect biometric information, such as the user's vital sign (including body temperature, pulse data, respiration data, and blood pressure). The sensor 124 can generate a signal based on the collected user information and transmit the signal to a processor that can cause an output 136 to perform an action in response to the signal (i.e., in response to the biometric information collected by the sensor 124. For example, a user may perform a rigorous activity, such as lifting weights or working out while wearing the HMD 100. During such activity, the user's heart rate or other vital signs may be elevated or changed and is detectable by the sensor 124. The sensor 124 can generate one or more signals based on the received input. The sensor 124 can transmit the signal to one or more components of the HMD 100 (e.g., to the display 108, to the output 136). The display 108, being in electrical communication with the facial interface 112, can receive electrical communication and provide feedback to the user related to their biometric readings (e.g., visual feedback, audio feedback, haptic feedback, etc.). Feedback can include determining when the user needs to take a break, or when the difficulty of an activity needs to be lowered or raised, the output can include scheduling various activities for the user or recommending adjustment of the HMD.

As used herein, the term “sensor” refers to one or more different sensing devices, such as a camera or imaging device, temperature device, oxygen device, movement device, brain activity device, sweat gland activity device, breathing activity device, muscle contraction device, etc. As used herein, the term “biometric,” “biometric information,” or “biometric feature” can refer to vital signs, including heart rate, pulse, respiration rate, respiration amplitude, or any other health related data. The term “biometric,” “biometric information,” or “biometric feature” can also refer to biological measurements or physical characteristics, such as facial expressions or emoting. In some examples, the sensor can detect or sense biometric features including features of the autonomic nervous system. Some particular examples of sensors include an electrooculography (EOG) sensor, electrocardiography (ECG or EKG) sensor, photoplethysmography (PPG) sensor heart rate sensor, heart rate variability sensor, blood volume pulse sensor, oxygen saturation (SpO2) sensor, compact pressure sensor, electromyography (EMG) sensor, core-body temperature sensor, galvanic skin response (GSR) sensor, functional near-infrared spectroscopy (FNIR) sensor, non-contact passive infrared (IR) sensor, accelerometer, gyroscope, magnetometer, inclinometer, barometer, infrared sensor, global positioning system sensor, etc. Additional sensor examples can include, contact microphones (e.g., pressure-based MEMS), bioelectrical activity sensors, UV exposure sensors, or particle sensors. In some examples, certain sensors can be used to assess stress and emotion. The HMD can then provide feedback or output related to the detected stress and emotion. In some examples, the sensors can operate through coin cell battery or Bluetooth connectivity. In some examples, the sensors are powered by a primary battery of the HMD.

The sensors described herein can allow for observations of the autonomic nervous system (ANS), to observe relaxation and stress indicators, mental health, medical treatments, etc. Using the sensors, physicians and care takers could have live feedback of biometrics. Use cases can include fitness settings, user content, workplace, telepresence, clinical, education, training, pain, therapy, etc. In some examples, the sensors can be used to capture facial expressions. This is particularly relevant given the user's face is covered by HMD. For example, the HMD could use MEMS or motion tracking sensors to detect facial expressions

Any of the features, components, and/or parts, including the arrangements and configurations thereof shown in FIG. 1 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 1.

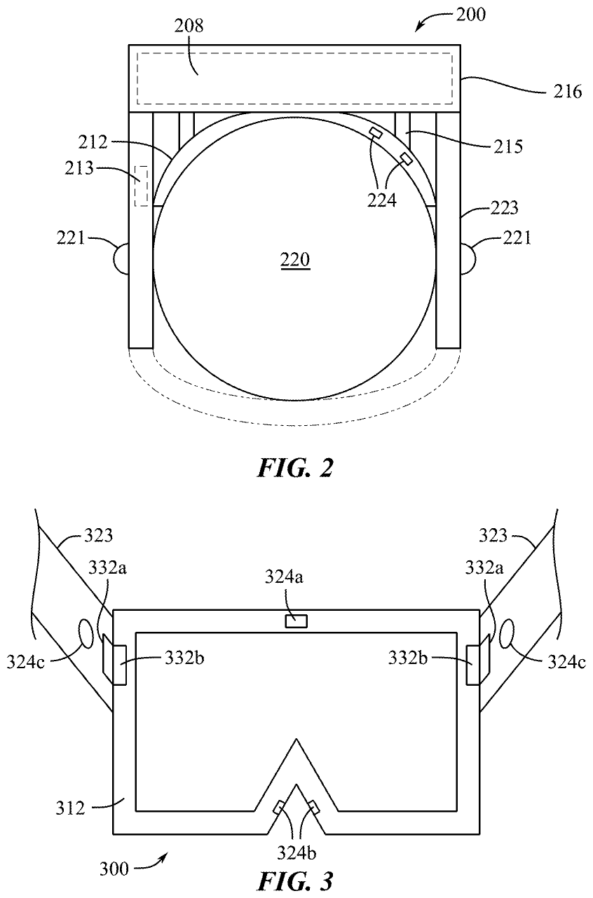

FIG. 2 illustrates a head-mountable device 200. The head-mountable device 200 can be substantially similar to, including some or all of the features of, the head-mountable devices described herein, such as head-mountable device 100. In some examples, the head-mountable device 200 includes a controller 213 (e.g., sensor controller). While the controller 213 is illustrated as being positioned in the support 223, the position of the controller 213 is not limited to the support 223, but can be positioned in/on the facial interface 212 or in the HMD display housing. The controller 213 can include a processor and a memory device storing computer-executable instructions that, when executed by the processor, cause the controller to receive biometric data from one or more sensors 224, transmit a signal based on the sensor data, and generate a signal to cause a component to perform an action in response to the signal.

The controller 213 can include one or more processors (e.g., a system on chip, integrated circuit, driver, microcontroller, application processor, crossover processor, etc.). Further, the controller 213 can include one or more memory devices (e.g., individual nonvolatile memory, processor-embedded nonvolatile memory, random access memory, memory integrated circuits, DRAM chips, stacked memory modules, storage devices, memory partitions, etc.). In certain implementations, the controller 213 is communicatively coupled to a power source (e.g., a battery).

In some examples, the controller 213 stores sensor data received from the sensor(s) 224 in the memory. The controller 213 can receive and/or transmit signals based on sensor data. For example, as will be described below, the controller 213, by way of the processor and memory, can transmit a signal to the display 208 based on the sensor data (e.g., causing the display 208 or the head-mountable device 200 to perform an action, such as present a certain message, power off, react to biometric feedback, etc.

The controller 213 can perform any number of different functions. For example, the memory device can store computer-executable instructions that, when executed by the processor, cause the controller to receive sensor data from the sensors 224 and transmit a signal based on the sensor data. For instance, the controller 213 can transmit a sensor signal to a display 208. In response to the controller 213 the display 208 can perform a wide variety of actions, including power off or power on, react to a user generated facial expression, present a digital notification (e.g., user-generated notification, push notification, context-generated notification, system-generated notification, smart notification, etc.). In some examples, the memory device storing computer-executable instructions that, when executed by the processor, cause the controller 213 to receive biometric data from the sensor(s) 224, transmit a signal based on the sensor data, and perform an action in response to the signal.

As illustrated, the head-mountable device (e.g., wearable electronic device) 200 can include the display 208, the facial interface 212, and the sensor(s) 224 coupled to the engagement interface 212. The sensors 224 can be embedded, encapsulated, deposited, adhered, or otherwise attached to the facial interface 212. The sensors 224 can be non-contact sensors or can directly contact or touch the user's head. The sensor(s) 224 can be configured to detect a biometric feature of the user 220. The sensor(s) 224, upon detecting a signal from the biometric feature, can transmit the signal to a component of the head-mountable device 200 causing the head-mountable device 200 to perform an action. For example, the head-mountable device 200 can change shape (i.e., tighten or loosen), move, vibrate, rotate, recalibrate, or reposition in response to the sensor signal of the biometric feature.

The head-mountable device 200 can include a support, headband, or retention band 223 connected to the display 208 and/or frame 216. The retention band 223 is configured to secure the display 108 and/or frame 216 relative to the user's head 220 (e.g., such that the display 208 is maintained in front of a user's eyes). The retention band 223 can be constructed from elastic material, inelastic material, or a combination of elastic and inelastic material. The retention band 223 is adjustable such that the retention band 223 conforms to the various shapes and sizes of a user's head 220. In some examples, the retention band 223 secures the head-mountable device 200 via friction between the user's head 220 and the retention band 223. In some examples, the retention band 223 elastically secures the head-mountable device 200 to the user's head 220. In some examples, the retention band 223 is coupled to a ratchet system or mechanism securing the head-mountable device 200 to the user's head 220. In some examples, the retention band 223 is disposed above or on an ear 221 of the user 220, supporting the head-mountable device 200.

In some examples, the housing or frame 216 of the head-mountable display 200 is connected via a connector 215 to the facial interface 212, the retention band 223 being connected to the frame 216 and/or the facial interface 212, securing the head-mountable device 200 to the user's head 220 above or over the ears 221 of the user.

Any of the features, components, and/or parts, including the arrangements and configurations thereof shown in FIG. 2 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 2.

FIG. 3 shows a rear view of a head-mountable device 300. The head-mountable device 300 can be substantially similar to, including some or all of the features of, the head-mountable devices described herein, such as head-mountable device 100 and 200. The head-mountable device 300 can include a facial interface (e.g., a facial engagement feature) 312 and a retention band 323. The head-mountable device 300 further includes a connectors 332a, 332b (collectively referred to as connector(s) 332) connecting the retention band 323 and the facial engagement interface 312. In some examples, the facial interface 312 is adjustable, accommodating to a user's head in a comfortable and secure way that shields the user from ambient light.

The connector 332 connecting the retention band 323 to the facial engagement interface 312 includes a first connector side 332a attached to the retention band 323 and a second connector side 332b attached to the facial engagement feature 312. In addition to forming a mechanical attachment between the retention band 323 and the facial interface 312, the connector 332 can include various types of electrical connections, such as a pogo-pin connection, or other connection types to electrically connect the retention band 323 with the facial interface 312. When mated, the first connector side 332a and the second connector side 332b mate (e.g., mechanically mate, magnetically mate via a magnet, electrically mate, etc.) such that the connectors 332 are in mechanical contact and electrical contact. When the first connector side 332a and the second connector side 332b are connected in this manner, electrical signals can be relayed through the from the retention band 323 to the facial engagement interface 312, and vice versa. That is, when connected, the facial engagement interface 312 can provide data and/or power to the retention band 323, for example to a sensor or electrical component 324c of the retention band. Likewise, when connected via connector 332, the retention band 323 can provide data and/or power to the facial interface 312, for example, to the sensors 324a and/or 324b. In some examples, the connectors 332a, 332b can magnetically mate or couple one to the other, transmitting information via a wireless communication connection and receiving power from a wireless power source (e.g., an inductive charger).

The head-mountable device 300 includes one or more sensor(s) 324a, 324b, 324c (collectively “sensors 324”). In some examples, the sensors 324 can be in contact (indirect or direct) with a user. For example, the sensors be positioned to receive input from a user's forehead, cheek, nose, temple region, back of the head, or at any location on the user's head/face. In some examples, the sensors 324 can be in indirect contact with a user. For example, the sensors 324 may detect a user's biometric features via infrared, IR, optics, imaging, or other means of detecting biometric features in an indirect or contactless way. The sensors can be a biometric or health sensor, such as any of those described above.

In some examples, the sensors 324 include a first sensor 324a oriented towards a first facial region (e.g., the forehead) and coupled to the engagement interface 312, a second sensor 324b oriented towards a second facial region (e.g., a nasal region) and coupled to the engagement interface 312, and a third sensor or sensor set 324c coupled to the retention band 323 and configured to contact a side or back of the user's head. When wearing the head-mountable device 300 the first sensor 324a orients towards the first facial region is different in orientation and location from the second sensor 324 which orients toward the second facial region. It will be appreciated by one skilled in the art that other configurations are contemplated herein, and the above example is for illustrative purposes only.

The sensors 324 can be located at various locations of the head-mountable device 300 and can differ in function or sensing capacity depending on where the sensor is located. For example, the sensors 324b are positioned on the engagement interface 312 proximate a nose of the user and may sense a user's respiratory biometrics providing a user with feedback indicating oxygen saturation (e.g., SPO₂). In some examples, the sensor 324a is positioned on the engagement interface 312 contacting a forehead of a user. In some examples, the sensor 324a detects pulse, facial expression, brain activity or stress response while performing an activity. In some examples, the sensor 324c is positioned on the retention band 323, adjacent the temple(s) of a user or at another location where the retention band 323 is contacting the user. In some examples, a sensor fusion exists in which one or more of the sensors 324 coordinate and communicate with each other and/or with other sensors on the HMD 300 to collectively gather user data.

In some examples, forehead sensors, such as sensor 324a, can be used for brain imaging and observations of the central nervous system. While a single forehead sensor 324a is shown in FIG. 3B, it will be understood that multiple sensors or a sensor array can be located on the facial interface 312 to contact, or be proximate to, the user's head. In some examples, the sensors can be configured to perform EEG (electroencephalography) or functional near-infrared spectroscopy (FNIR).

In some examples, the position of the forehead sensors 324a can be tuned or tailored to sense brain areas related to language, learning, memory, comprehension, sleep, stress, pain, attention, fear, discomfort, etc. In some examples, the facial interface 312 can be integrated with a sensor array that includes one or more transmitters that emit a signal. The transmitter(s) can be positioned a certain distance away from one or more detectors that sense the emitted signal. Based on the received signals by the detectors, a processor can infer or determine certain brain activity. In some examples, the sensors 324a can form a brain-computer interface (BCI), such as a non-invasive neural interface. In some examples, the integrated sensor array can be used to detect the parieto-frontal network of the brain.

In some examples, the sensors 324a, 324c can be used to perform EEG detections. The sensors 324a, 324c can measure the electrical activity in the cerebral cortex (the outer layer of the brain). The sensors 324a, 324c can include electrodes that are placed on a user's head, then the electrodes can non-invasively detect brainwaves from the subject. The EEG sensors 324a, 324c can record up to several thousands of snapshots of the electrical activity generated in the brain every second. The recorded brainwaves can be sent to amplifiers, then to a local processor, a remote electronic device, or the cloud to process the data.

In some examples, the sensors 324a, 324c can be used to perform functional near-infrared spectroscopy (FNIR). The sensors 324a, 324c can use low levels of non-ionizing light to record changes in cerebral blood flow in the brain through optical sensors placed on the surface of the head.

The sensors described herein can perform both passive and active EEG. The passive electrode signals can be sent to a remote processor to analyze. Amplification of the active EEG signals can be performed locally at the site to eliminate coexistence and interference that may be present with passive signal.

The HMD can include an output or feedback module that provides feedback based on the detections from the sensors. The feedback from the system can include displaying visualizations to the user. For example, the feedback can include breathing visualizations, visualization that relate to the user's attention, promptings or suggestions to the user to take a break or change an activity, or make other recommendations or notifications to the user.

Any of the features, components, and/or parts, including the arrangements and configurations thereof shown in FIG. 3 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 3.

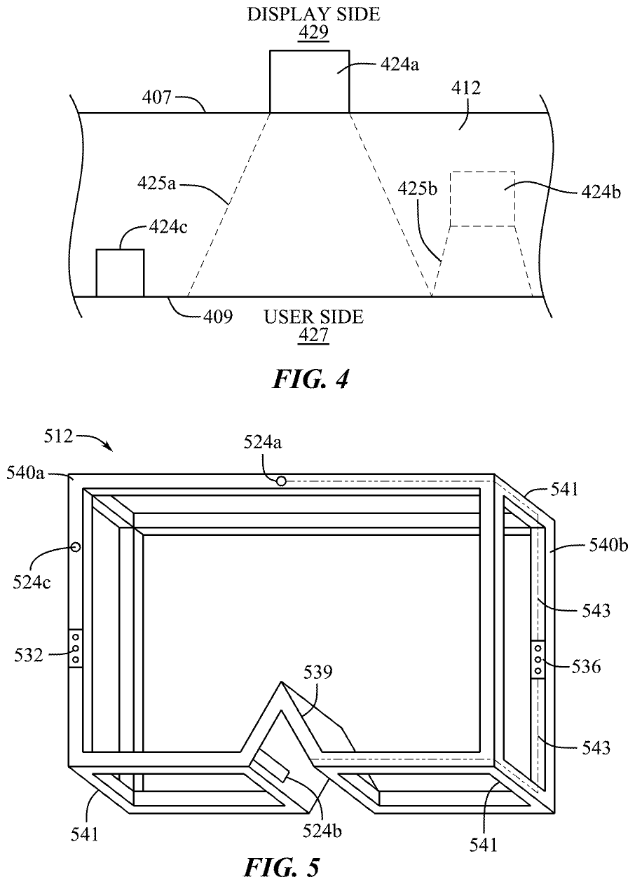

FIG. 4 shows a side or cross-sectional view of portion of a facial interface 412 wherein a sensor(s) 424 is embedded in the facial interface 412. The facial interface 412 includes a first side or surface 407 facing the display side 429 (i.e., toward the HMD display) and a second side or surface 409 facing toward a user side 427. In some examples, the first surface 407 attaches to a frame of the HMD. In some examples, the second surface 409 directly contacts or touches the user's head when the HMD is donned or being worn by the user.

The facial interface 412 can include one or more sensors 424a, 424b, 424c (collectively “sensors 424). The sensor 424a can be positioned on the first surface 407 of the facial interface 412. In some examples, the first surface 409 can an interior and the second surface 409 can be an exterior of the facial interface 412. Thus, the sensor 424a can be positioned on an interior of the facial interface 412. In some examples, the facial interface 412 can include a sensor 424b is embedded, encapsulated, or otherwise surrounded by the facial interface 412. The facial interface 412 can include, a sensor 424c that positioned such that a portion of the sensor 424c is exposed through the second surface 409. In other words, the sensor 424c can at least partially define the second surface 409 and can directly contact or touch the user's skin. In some examples, the sensor 424c can be partially surrounded or embedded in the facial interface 412. In some examples, the sensor 424c is external to the facial interface 412, positioned on the exterior 409 of the facial interface 412.

The sensors 424a and 424b can have corresponding sensor areas 425a, 425b, respectively. The sensor areas 425a, 425b can represent a field of view or cone of influence that is transparent to a signal emitted by the sensors 424a, 424b. In some examples, the field of view of the sensors 424 can be approximately 50 degrees. The sensors 424a, 424b can detect physiological, biological, and/or biometric changes of the user's body through corresponding sensor areas 425a, 425b. For example, the sensors 424a, 424b can detect changes to a user through the material of the facial interface 412. Other sensors may be added or substituted, as may be desired. In some examples, the sensor areas 425a, 425b can comprises a different material than the rest of the facial interface 412. For example, the sensor areas 425a, 425b can be transparent to certain signals from the sensors 424a, 424b or from the user, while the remainder of the facial interface 412 is not transparent or transmissive to such signals.

Sensor 424b is a distance from the first surface 407, disposed with in the facial interface 412, and therefore closer to the second surface 409 than the sensor 424a. In some instances, this closer positioning of the sensor 424b to the second surface 409 can correspondingly reduce the field of view 425b. In some examples, the sensor 424c may be flush with the second surface 409 and/or directly contacting a user's face and/or skin. This is only one example of sensor depth variation, as a plurality of sensors can be disposed on the first surface 407 of the facial interface 412, or between the first surface 407 and the second surface 409 of the facial interface 412. In some examples, the second surface 409 (e.g., user side 427) abuts a forehead region or nasal region of a user head when the head-mountable device 100 is donned.

Any of the features, components, and/or parts, including the arrangements and configurations thereof shown in FIG. 4 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 4.

FIG. 5 shows a front perspective view of a facial interface 512. The facial interface 512 can be substantially similar to, including some or all of the features of, the facial interface described herein, such as facial interfaces 112, 212, 312, and 412. In some examples, the facial interface 512 can include a framed or clamshell design. In some examples, various components and/or circuitry can be discretely positioned within the volume of the clamshell design. The facial interface 512 can include a first side 540a and a second side 540b. The first side 540a can be proximate the user when donned. In some examples, the first side 540a can contact the user's face. The second side 540b can be positioned between the first side 540a and a display of the HMD. In some examples, the second side 540b is attached to the display housing.

The first side 540a can include a connector 532 configured to attach the facial interface 512 to a component, such as a retention band. The connector 532 can be mechanical and/or electrical. In some examples, the connector 532 is electrically connected to a sensor 524c. The first side 540a can configured to contact a face of a user. The first side 540a can include sensors 524a, 524b, 524c, 524d that can be configured to detect a biometric feature(s) of the user. In some examples, the facial interface includes a nasal support 539. The nasal support 539 can be configured to contact the user's nose. The nasal support 539 can include at least one sensor 524b.

In some examples, the facial interface 512 can be interchangeable with a different facial interface having different sensors or structure. For example, a user may exercise with the head-mountable device 100 using a first facial interface with specific sensors for exercising. The same user then may game or perform a leisure activity using the head-mountable device 100 fitted with a different facial interface with different sensors or structure for those activities. This is only one example and one of ordinary skill in the art will appreciate other examples are contemplated herein.

In some examples, the sensors 524 are removably attached to the facial interface 512. For example, a user may wish to replace or add certain sensors to the facial interface 512. A user can selectively attach or remove a sensor 524 and replace it with a different sensor depending on the activity of the user or purpose of the sensor. In this way, the sensors can accommodate the needs or demands of a user for a specific activity requiring specific sensors without needing to replace the entire HMD or facial interface 512.

In some examples, the facial interface 512 includes a connector 536. The connector 536 can be electrically connected one or more of the sensors 524 via a wired or wireless connection 543. In some examples, the facial interface 512 is in electrical communication with a display via the wired connection link 543 and/or the connector 536, such as, a braided electrical cable, a pogo connection, or other type of wired connection. In some examples, the facial interface 512 can be in electrical communication with the display 208 via a wireless connection (e.g., low-power Bluetooth BLE connection). In some examples, data or signals from the sensors 524 are transmitted through the link 543 to the connector 536, where they are further transmitted to the HMD itself to be processed. Thus, in some examples, the facial interface 512 serves as an interconnect between the retention band and the display unit. In some examples, the retention band can mate directly into the display unit, bypassing the facial interface 612. In some examples, the facial interface 512 can include an Orion style connection (e.g., 3 contact pads and 3 contact pins).

In some examples, the facial interface 512 includes a bridging feature 541 structurally connecting the first side 540a and the second side 540b. In some examples, the bridging features 541 can provide a conduit for wires or electronics, such as the link 543. In some examples, the bridging feature 541 is flexible permitting the facial interface first side 540a to move, pivot, or change shape relative to the facial interface second side 540b. In some examples, the bridging feature 541 is rigid maintaining the facial interface first side 540a unmoved relative to the facial interface second side 540b.

Any of the features, components, and/or parts, including the arrangements and configurations thereof shown in FIG. 5 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 5.

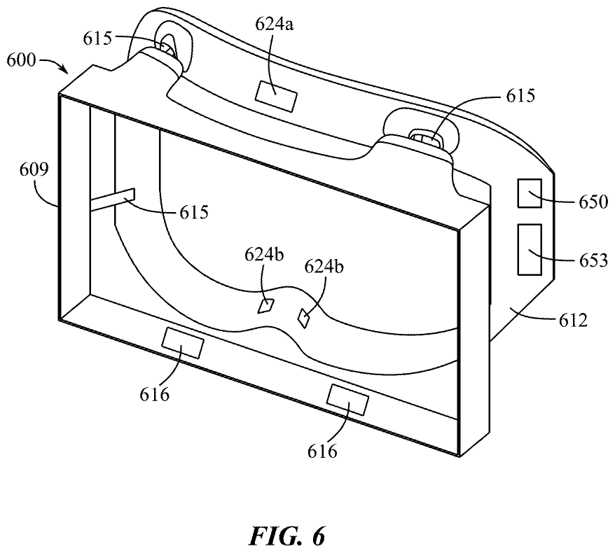

FIG. 6 illustrates a perspective view of select components of a head-mountable device 600. The HMD 600 can be substantially similar to, including some or all of the features of, the HMDs described herein. The HMD 600 can include a chassis or frame 609 that can at least partially house a display, and a facial interface 612 that can contact a user's face. In some examples, the HMD 600 changes shape in response to a signal from one or more sensor(s) 624a, 624b, 624c (collectively “sensors 624”). The frame 609 can be connected via a connector(s) 615 to the facial interface 612. In some examples, the connectors 615 are electrical and/or mechanical connectors configured to establish electrical and/or mechanical connections between the frame 609 and the facial interface 612. In some examples, the connectors 615 are articulable or movable, such that the connectors 615 can move to change a shape of the HMD 600. In some examples, the HMD 600 can include strain gauges or point load pressure sensors that can be used on posts 615 to act as pressure sensors to extrapolate retention band tension. The system can then guide the user to adjust for proper fitting of the HMD (e.g., prompt to tighten or loosen or change an angle of the band. In some examples, the display can illustrate for the user how to adjust band up/down based on the detected tension. In some examples, semiconductor(s) can be embedded in the facial interface (e.g., in the foam of the facial interface).

In some examples, the facial interface 612 can be integrated with one or more feedback modules or outputs 650, 653. The sensors 624 can receive biometric input from a user. Based on the input detected by the sensors 624, the outputs 650, 653 can perform some action. The outputs 650, 653 can include LEDs, haptics, speakers, motors, displays, or any other feedback devices that creates an output in response to the signals of biometric information from the sensors 624. For example, one or more of the sensors 624 can detect when the HMD is donned, for example through capacitive or pressure sensors. The sensors 624 can then generate a signal to be analyzed by a processor. The processor can then cause a first feedback output 650, such as an LED to emit a certain color of light indicating that the head-mountable device 600 is donned. In some examples, the second feedback output 653 can be a feedback output such as a haptic feedback output alerting a user of a notification (e.g., message notification, text notification, email notification, etc.) In some examples, the output 653 can be a display visible by the user and which can extend the peripheral view of the user. It should be appreciated by one of normal skill in the art that the above example is one example and other examples of feedback outputs are contemplated herein.

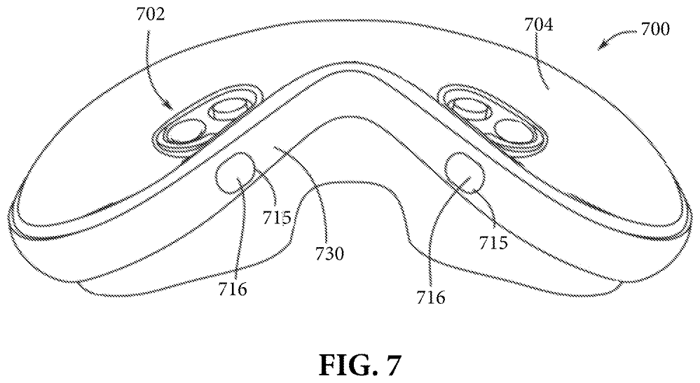

In some examples, the chassis or frame 609 can at least partially define an exterior surface of the HMD 600. In some examples, health sensors 616 can be mounted, attached, or otherwise integrated into/onto the chassis 609. While two health sensors 616 are illustrated, it will be understood that one, two, or more than two sensors can be used and are contemplated by the present disclosure. The health sensors 616 can be substantially similar to, including some or all of the features of, the sensor described herein (e.g., sensors 624). In some examples, the health sensors 616 can be non-contact sensors (i.e., the health sensors 616 can collect the intended data without needing to be in direct physical contact with the user. The health sensors 616 can be positioned on a bottom surface of the chassis 609. The health sensors 616 can at least partially define an exterior surface of the HMD 600. Further details of chassis-mounted health sensors are described below, with reference to FIG. 7.

Any of the features, components, and/or parts, including the arrangements and configurations thereof shown in FIG. 6 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 6.

FIG. 7 illustrates a bottom perspective view of an example of an HMD 700 including a front display/cover assembly 704, forward facing sensors 702, and user-facing, chassis-mounted health sensors 716. The health sensors 716 can be similar to other sensor systems described above, including in reference to FIG. 6. In at least one example, the health sensors 716 can be facing at least partially downward and inward to capture images of the user's lower facial features. In one example, the health sensors 716 can be coupled directly to the frame, chassis, or housing 730, or to one or more internal brackets directly coupled to the frame or housing 730 shown. The frame or housing 730 can include one or more apertures/openings 715 that receive the health sensors 716 and through which the health sensors 716 can send and receive signals.

In some examples, the health sensors 716 can include cameras. The inward facing cameras can capture images of the user when the user dons the head mountable device 700. When the HMD 700 is worn by a user, the health sensors 716 can be directed toward a nose and/or mouth of the user. The health sensors 716 can include infrared (IR) sensors (passive and/or active), proximity sensors, motion sensors, moisture sensors, carbon dioxide sensors, or any other suitable non-contact sensor for gathering information related to the user's face, mouth, and nose. In some examples, at least one of the health sensors 716 can be an IR sensor aimed at the nostrils of the user and configured to detect thermal changes caused by the user breathing. In some examples, at least one of the health sensors 716 can be a proximity sensor capable of detecting slight movements of the nostrils (nostril flare) caused by the user's breathing. In this manner, the health sensors 716 can act as respiration sensors and can assist in observing the user's respiratory operation, as described herein. In some examples, the health sensors 716 can include depth cameras that can monitor key landmarks around the user's face. In some examples, the health sensors 716 can include a camera having a wide field of view sufficient to perform both body tracking and respiratory tracking. In other examples, the HMD 700 includes a chassis-mounted camera dedicated to respiratory tracking.

By incorporating the health sensors 716 directly into/onto the chassis 730, the health sensors 716 can be electrically connected directly to a processor or controller of the HMD 700. For example, a flex cable or flex arm can act as a direct interconnect to establish an electrical connection between the health sensors 716 and the various hardware components of the HMD (e.g., main logic board). Advantageously, the configuration removes a need for an interconnect travelling between the facial interface and the HMD housing 730.

While FIG. 7 illustrates only one health sensor 716 positioned in each opening 715, it will be understood that multiple sensors can be positioned in a single opening 715. In some examples, the sensors 716 can at least partially be disposed within an internal volume that is at least partially defined by the housing 730. In some examples, the sensors 716 are mounted to the outside of the housing 730, such that no portion of the sensors is disposed within an internal volume defined by the housing 730. In such examples, the housing can define an opening for wiring to pass between the sensors 716 and the internal components of the HMD 700. In some examples, electrical terminals or interconnects can be positioned directly at the exterior surface of the housing 730 such that the sensors directly “plug” into the terminals from the exterior of the housing 730.

Any of the features, components, and/or parts, including the arrangements and configurations thereof shown in FIG. 7 can be included, either alone or in any combination, in any of the other examples of devices, features, components, and parts described herein. Likewise, any of the features, components, and/or parts, including the arrangements and configurations thereof shown and described with reference to FIGS. 1-6 can be included, either alone or in any combination, in the example of the devices, features, components, and parts shown in FIG. 7.

To the extent the present exemplary systems and methods use personally identifiable information, such use of personally identifiable information should follow privacy policies and practices that are generally recognized as meeting or exceeding industry or governmental requirements for maintaining the privacy of users. In particular, personally identifiable information data should be managed and handled so as to minimize risks of unintentional or unauthorized access or use, and the nature of authorized use should be clearly indicated to users.

The foregoing description used specific, though exemplary, nomenclature to provide a thorough understanding of the described embodiments. The specific details are not required in order to practice the described examples. Thus, the foregoing descriptions of the specific embodiments and examples described herein are presented for purposes of illustration and description only. They are not intended to be exhaustive or to limit the embodiments to the precise forms disclosed. Many modifications and variations are possible in view of the above teachings.

What is claimed is:

1. A head-mountable device, comprising:
a frame;
a display positioned in the frame;
a processor; and
a facial interface connected to the frame, the facial interface comprising:
a sensor electrically coupled to the processor, the sensor configured to:
detect biometric information from a nasal region of a user; and
generate a signal based on the biometric information.

2. The head-mountable device of claim 1, wherein:
the sensor transmits the signal to the processor;
the processor analyzes the signal; and
the processor causes the head-mountable device to perform an action in response to the signal.

3. The head-mountable device of claim 2, wherein the action comprises generating at least one of a visual feedback, an audio feedback, or a haptic feedback.

4. The head-mountable device of claim 1, wherein the sensor comprises at least one of a temperature sensor, a respiration sensor, a stress response sensor, or a heart activity sensor.

5. The head-mountable device of claim 1, wherein the processor determines a facial expression of the user based on the signal.

6. The head-mountable device of claim 1, wherein the facial interface is removably attached to the frame via a magnet.

7. The head-mountable device of claim 1, wherein the sensor is removably attached to the facial interface.

8. The head-mountable device of claim 1, wherein the facial interface is in electrical communication with the display.

9. The head-mountable device of claim 1, further comprising a retention band;
wherein the facial interface comprises a connector to attach to the retention band.

10. A facial interface for a head-mountable device, comprising:
a first side comprising a connector configured to attach the facial interface to a display;
a second side configured to contact a face of a user; and
a sensor configured to detect a facial expression of the user; and
wherein the sensor is positioned proximate a nose of the user.

11. The facial interface of claim 10, wherein:
the sensor is embedded in the facial interface; and
the second side of the facial interface comprising an area transparent to a signal emitted by the sensor.

12. The facial interface of claim 10, further comprising a controller, the controller comprising:
a processor configured to:
receive a signal from the sensor indicative of the detected facial expression; and
analyze the signal; and
a memory device storing computer-executable instructions that, when executed by the processor, cause a component of the head-mountable device to perform an action in response to the signal.

13. The facial interface of claim 10, wherein the display performs a function based on the detected facial expression.

14. A wearable electronic device comprising:
a display;
an engagement interface; and
a sensor coupled to the engagement interface, the sensor configured to detect a biometric feature and produce a signal based on the biometric feature; and
a processor configured to analyze the signal;
wherein, in response to the signal, the wearable electronic device performs an action.

15. The wearable electronic device of claim 14, wherein the action comprises providing a notification to a user.

16. The wearable electronic device of claim 14, wherein:
the wearable electronic device comprises a head-mountable display; and
the engagement interface is adjustable.

17. The wearable electronic device of claim 14, the biometric feature comprises a feature of the autonomic nervous system.

18. The wearable electronic device of claim 14, wherein:
the sensor comprises a first sensor oriented towards a first facial region; and
further comprising a second sensor coupled to the engagement interface, the second sensor oriented towards a second facial region different from the first facial region when the wearable electronic device is worn.

19. The wearable electronic device of claim 14, wherein the engagement interface changes shape in response to the signal.

20. The wearable electronic device of claim 14, wherein:
the engagement interface is a first engagement interface;
the sensor is a first sensor; and
the display is removably attachable to the first engagement interface and a second engagement interface having a second sensor.
